(19) United States
(12) Patent Application Publication
(10) Pub. No.: US 2009/0043292 A1
(43) Pub. Date: Feb. 12, 2009

(54) Method for Preventing and Correcting Presbyopia

(76) Inventor: Iku Kuo, Thaipao City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/238,574

(22) Filed: Sep. 26, 2008

Related U.S. Application Data
(63) Continuation-in-part of application No. 11/836,272, filed on Aug. 9, 2007.

(51) Int. Cl. A61F 9/007

(52) U.S. Cl. 606/5

(57) ABSTRACT

A method for preventing and correcting presbyopia includes steps of forming a refractive correction surface for treating myopia, hyperopia or astigmatism to improve vision of distant objects in a central portion of the cornea of an eyeball corresponding in size and position to the pupil of the eyeball under a normal lighting condition; and successively forming lens-like refractive correction surfaces adaptive to presbyopia of different degrees in another portion of the cornea corresponding in size and position to the pupil dilating to the largest extent excluding an area occupied by the pupil under the normal lighting condition. Thus, when a person treated with the method grows older and experiences degenerate eye functions, the pre-formed lens-like refractive correction surfaces having different curvatures can correct presbyopia of different degrees at different ages and adequately improve vision of nearby objects, thereby achieving the intended effect of preventing presbyopia.

### BACKGROUND OF THE INVENTION

1. Technical Field

The present invention relates to a method for preventing presbyopia through refractive treatments, in which refractive correction surfaces of different curvatures are formed on different portions of a cornea to not only solve existing vision problems, but also correct vision problems attributed to presbyopia when eye functions degenerate.

2. Description of Related Art

As shown in FIG. 1, a cornea 2 of a normal eyeball 1 deflects incoming light through a pupil 3 of the eyeball 1. Under action of an iris 5, the pupil 3 dilates or constricts to regulate the amount of light passing therethrough. Then the light is refracted by a crystalline lens 4 of the eyeball 1 so as to focus on a retina 6 and thereby produce an image formed thereon, before the image is transmitted to the brain via an optic nerve 7. The shape of the cornea 2 is very important because the cornea 2 carries out over two thirds of the focusing function of the eyeball 1. The cornea 2, when functioning properly, can deflect incoming light correctly and allow an image to focus clearly on the retina 6. However, if the cornea 2 or the crystalline lens 4 fails to regulate light properly, a blurry vision will occur as images no longer focus accurately on the retina 6.

For example, myopia refers to a condition in which light entering the eyeball 1 is refracted so as to focus in front of the retina 6, as shown in FIG. 2. As a result, distant objects cannot be seen with clarity. If the light entering the eyeball 1 is refracted so as to focus behind the retina 6, as shown in FIG. 3, hyperopia, or better known as far-sightedness, arises. In addition, a non-uniform spherical curvature of the cornea 2 results in astigmatism, in which case the light entering the eyeball 1 has multiple foci, as shown in FIG. 4. Moreover, when the elasticity of the crystalline lens 4 of the eyeball 1 gradually dwindles or the contraction ability of the ciliary muscles deteriorates with age, the focusing function of the eyeball 1 becomes poorer, leading to a blurry vision of nearby objects, which is called presbyopia. It should be noted that when people with myopia grow older, they are still susceptible to presbyopia as their eye functions degenerate.

All the abovementioned diseases resulting from abnormal focusing in the eyeballs are generally referred to as refractive errors, which may also be caused by an abnormal shape of the eyeball 1 or dysfunction of the crystalline lens 4. In order to compensate for or improve the focusing ability of the eyeballs, myopia can now be corrected by excimer laser-assisted surgery in addition to the conventional vision correction methods such as wearing glasses or contact lenses. The excimer laser-assisted surgery is performed by using a laser to vaporize a thin layer of tissues 20 in a central portion of the cornea 2 so as to flatten the curvature of that portion of the cornea 2, as shown in FIG. 5. Capable of correcting myopia of dioptric values ranging from −1.0 to −20.0 with high precision, the excimer laser-assisted surgery neither reduces the robustness of the eyeballs nor produces such sequelae as nyctalopia and hemeralopia, thereby significantly increasing the safety and precision of myopia correction. Besides, the excimer laser-assisted surgery is also applicable to the correction of hyperopia and astigmatism.

People having received such surgery may well expect a life of improved eyesight without wearing glasses in the following five to ten years, even though their eyeballs degenerate in terms of regulatory functions as a result of aging. However, as these people grow older, presbyopia may develop to higher degrees, and the same is true of hyperopia and astigmatism.

### BRIEF SUMMARY OF THE INVENTION

Therefore, in order to prevent people having received the foregoing surgery for vision correction from having presbyopia, the present invention provides a method comprising steps of forming a refractive correction surface for improving vision of distant objects in a central portion of the cornea of an eyeball corresponding in size and position to the pupil of the eyeball under a normal lighting condition; and further forming successive lens-like refractive correction surfaces having predetermined curvatures in a portion of the cornea having a predetermined range outside and surrounding the central portion.

Thus, according to the present invention, after the lens-like refractive correction surfaces having the predetermined curvatures for correcting presbyopia of different degrees are successively formed on the predetermined portion of the cornea in an outward direction, not only can existing vision problems be corrected, but also presbyopia can be prevented by the pre-formed wavy refractive correction surfaces, which provide adequate improvement in vision of nearby objects, so that vision correction and presbyopia prevention are simultaneously achieved.

### BRIEF DESCRIPTION OF THE SEVERAL VIEWS OF THE DRAWINGS

The invention as well as a preferred mode of use, further objectives and advantages thereof will be best understood by referring to the following detailed description of an illustrative embodiment in conjunction with the accompanying drawings, wherein:

FIG. 1 is a schematic illustration of an eyeball having normal eyesight;

FIG. 2 is a schematic illustration of an eyeball having myopia;

FIG. 3 is a schematic illustration of an eyeball having hyperopia;

FIG. 4 is a schematic illustration of an eyeball having astigmatism;

FIG. 5 is a schematic illustration of an eyeball having received a conventional laser-assisted surgery;

FIG. 6 is a schematic illustration of an eyeball of a person with myopia after being treated by a method of the present invention;

FIG. 7 is an enlarged view of a cornea of the eyeball in FIG. 6 after being treated by the method of the present invention;

FIG. 8 is a schematic illustration of an eyeball of a person with hyperopia after being treated by the method of the present invention; and

FIG. 9 is an enlarged view of a cornea of the eyeball in FIG. 8 after being treated by the method of the present invention.

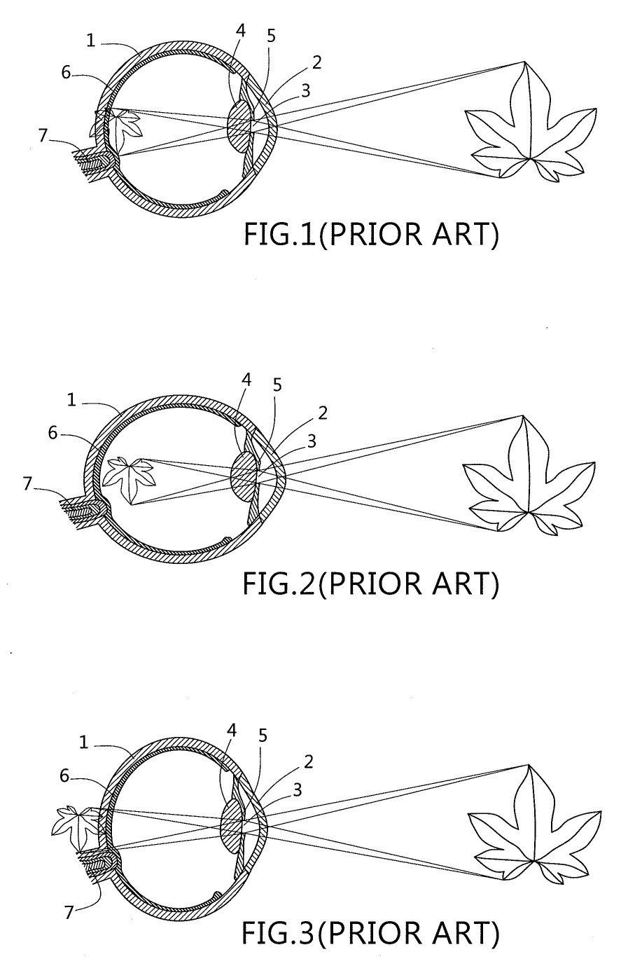

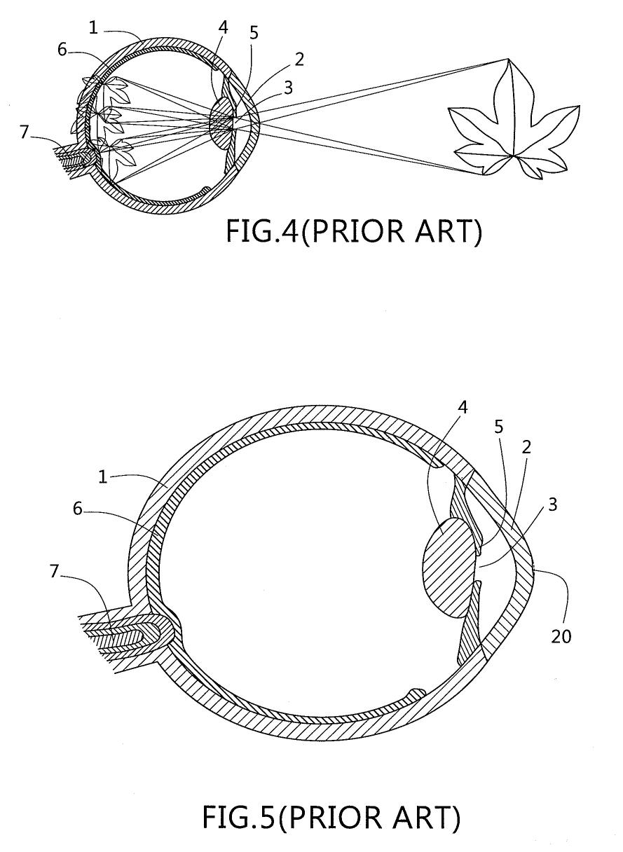

### DETAILED DESCRIPTION OF THE INVENTION

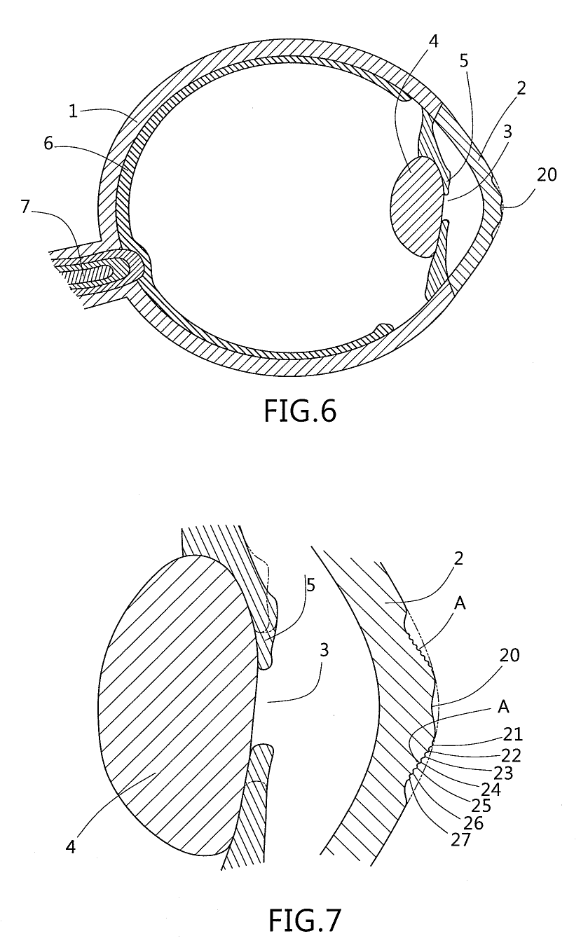

Referring to FIGS. 6 and 7, a person with myopia has eyeballs whose optic axes are excessively long or whose corneas and crystalline lenses have excessively large deflection angles. Therefore, according to the present invention, a refractive correction surface 20 having a predetermined curvature is formed on a central portion of a cornea 2 of an eyeball 1 corresponding in size and position to a pupil 3 of the eyeball 1 under a normal lighting condition. Then, a concavely curved surface A having a predetermined curvature is formed on the cornea 2, extending outward from the central portion of the cornea 2. Afterward, a plurality of continuous lens-like refractive correction surfaces 21 to 27 are successively formed on the surface A in an outward direction. Each of the refractive correction surfaces 20 to 27 is configured to project an image to a different location in front of a retina 6 of the eyeball 1. For example, the central refractive correction surface 20 is configured to provide vision correction according to current dioptric values of myopia, while the other refractive correction surfaces 21 to 27 are configured to prevent presbyopia by providing adequate improvement and correction in vision of nearby objects. For example, in a case where a 50-year-old person has minor presbyopia that leads to slightly blurry view of nearby objects, the refractive correction surface 21 may serve to correct the current vision problem by projecting an image right on the retina 6, so as to provide vision of nearby objects with increased clarity. On the other hand, the other refractive correction surfaces 22 to 27 may be applicable to presbyopia of different degrees at different ages, respectively.

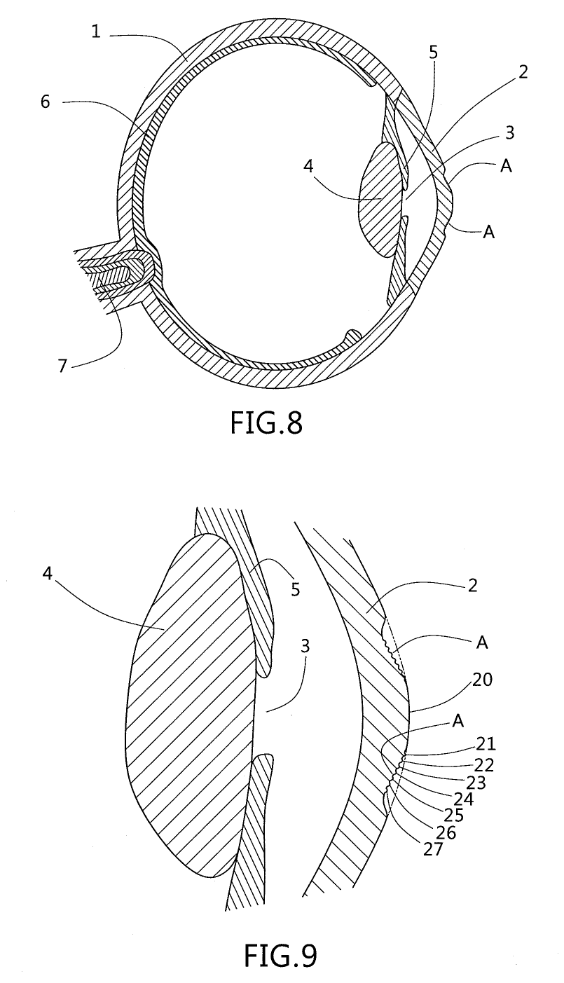

In addition, as shown in FIGS. 8 and 9, the method of the present invention is also applicable to a person with hyperopia. More specifically, the refractive correction surface 20 having a predetermined curvature is formed on a central portion of the cornea 2 of the eyeball 1 corresponding in size and position to the pupil 3 of the eyeball 1 under a normal lighting condition. Following that, a concavely curved surface A having a predetermined curvature is formed on the cornea 2, extending outward from the central portion of the cornea 2. Then, a plurality of continuous lens-like refractive correction surfaces 21 to 27 are successively formed on the surface A in an outward direction. Each of the refractive correction surfaces 20 to 27 is configured to project an image to a different location in front of the retina 6 of the eyeball 1, thereby achieving the purpose of preventing and correcting presbyopia.

Furthermore, the method of the present invention is equally applicable to a person with astigmatism, except that a refractive correction surface having a predetermined curvature for correcting astigmatism is formed in a central portion of the cornea corresponding in size and position to the pupil under a normal lighting condition. Then, a plurality of lens-like refractive correction surfaces having predetermined curvatures are successively formed on the cornea, extending from the central portion thereof in an outward direction. Thus, presbyopia prevention and correction can also be achieved.

If the method of the present invention is performed only to solve vision problems associated with presbyopia, a concavely curved surface A having a predetermined curvature is formed in an outward direction in area overlapping a predetermined portion of the cornea 2 not corresponding in size and position to the pupil 3 under a normal lighting condition and a portion of the cornea 2 corresponding in size and position to the pupil 3 dilating to the largest extent. Then, a plurality of continuous lens-like refractive correction surfaces 21 to 27 are successively formed on the surface A in an outward direction, so as to prevent as well as correct presbyopia.

1. A method for preventing and correcting presbyopia, comprising a step of forming a plurality of lens-like refractive correction surfaces in a first portion of a cornea of an eyeball outside a second portion of the cornea corresponding in size and position to a pupil of the eyeball under a normal lighting condition.

2. The method for preventing and correcting presbyopia as claimed in claim 1, wherein the lens-like refractive correction surfaces are configured to correct presbyopia of different degrees, respectively.

3. The method for preventing and correcting presbyopia as claimed in claim 1, wherein the refractive correction surfaces are formed in an area overlapping the first portion of the cornea and a third portion of the cornea corresponding in size and position to the pupil dilating to a largest extent.

4. The method for preventing and correcting presbyopia as claimed in claim 1, wherein the lens-like refractive correction surfaces are concavely curved surfaces having predetermined curvature.

5. The method for preventing and correcting presbyopia as claimed in claim 1, further comprising a step of forming a refractive correction surface for treating myopia, hyperopia or astigmatism in the second portion of the cornea corresponding in size and position to the pupil under the normal lighting condition.
